(19) United States
(12) Patent Application Publication
(10) Pub. No.: US 2020/0093645 A1
(43) Pub. Date: Mar. 26, 2020

(54) Ear-Protective Headgear

(71) Applicant: Sherry Wallace, Quitman, MS (US)

(72) Inventor: Sherry Wallace, Quitman, MS (US)

(21) Appl. No.: 16/581,739

(22) Filed: Sep. 24, 2019

Related U.S. Application Data
(60) Provisional application No. 62/735,693, filed on Sep. 24, 2018.

(51) Int. Cl. A61F 11/14

(52) U.S. Cl. CPC A61F 11/14; A41D 20/00

(57) ABSTRACT

An ear-protective headgear has a headband, a first earpiece and a second earpiece. The headband is worn on the user's head in order to position the earpieces over the user's ears, protecting the ears from environmental discomforts, such as the heat from a hooded hair dryer. The earpieces may be moved along the headband to be appropriately positioned over the ears. The headband is also adjustable in length through a headband fastener connecting a first end and a second end of the headband together. The earpieces are affixed to the headband by two strap portions forming a headband connector, the ends of which are connected together, forming a loop around the headband. An earpiece connector insert may be positioned within the headband connector of one of the earpieces and attached to a fastening anchor positioned on the other earpiece, connecting the earpieces together to wear as an accessory.

The current application claims a priority to the U.S. Provisional Patent application Ser. No. 62/735,693 filed on Sep. 24, 2018.

### FIELD OF THE INVENTION

The present invention relates generally to head wearable accessories. More particularly, the present invention relates to headgear for protecting the ears from heat.

### BACKGROUND OF THE INVENTION

Hair care is an integral part of most people's lives. From getting a simple haircut to getting an extensive hair treatment, people's schedules are generally filled with a timeslot dedicated to a hair care-related activity. A common process utilized in various hair-related activities and treatments involves drying the hair of the user. Whether the drying is done after washing the hair or to help accelerate the setting of pin curls, hair drying is an important step in professional and amateur hair treatments and activities. A traditional hair drying apparatus utilized in the industry has been the hooded or bonnet hair dryer. In general, hooded hair dryers comprise a hollow, helmet-like structure with an integral heating mechanism which is positioned over the head of the user to dry the whole head. While not as commonly used as before, hooded hair dryers are still used today thanks to the even heat distribution hooded hair dryers can provided as compared to the handheld hair dryers. However, the slow drying and discomfort of using hooded hair dryers is major drawback. Due to the hooded hair dryer covering almost the entirety of the user's head, a common complaint is the heating of the ears during the hair drying. A hair cap is often used along with hooded hair dryers which can help alleviate the discomfort from the heat during the hair drying process. However, most currently available hair caps do not provide enough protection to the ears in order to reduce the overall discomfort while using a hooded hair dryer. Thus, a hair accessory which can be utilized along with a hooded hair dryer in order to protect the ears and surrounding area from the heat is beneficial and necessary.

An objective of the present invention is to provide a protective headgear, preferably referred to as “Hot Ears.” The protective headgear comprises a structure which is worn like a head piece. The protective headgear further comprises a flexible structure designed so the protective headgear can be stretched to fit the head of the user. The protective headgear comprises a pair of earpieces which move about to protect the ears from the heat while the user sits under a hooded hair dryer. The pair of earpieces of the protective headgear further comprises a trim around the edges. The trim around the pair of earpieces further comprises an adjustable closure which can be opened and closed.

Additional advantages of the invention will be set forth in part in the description which follows, and in part will be obvious from the description, or may be learned by practice of the invention. Additional advantages of the invention may be realized and attained by means of the instrumentalities and combinations particularly pointed out in the detailed description of the invention section. Further benefits and advantages of the embodiments of the invention will become apparent from consideration of the following detailed description given with reference to the accompanying drawings, which specify and show preferred embodiments of the present invention.

### BRIEF DESCRIPTION OF THE DRAWINGS

FIG. 1 is a disassembled perspective view of the present invention.

FIG. 2 is a perspective view of the present invention with the first earpiece and second earpiece attached to the headband.

FIG. 3 is a right raised perspective view of the earpieces and the earpiece connector insert in a disassembled state.

FIG. 4 is a left lowered perspective view of the earpieces and the earpiece connector insert in a disassembled state.

FIG. 5 is a right lowered perspective view of the earpieces being attached together by the earpiece connector.

FIG. 6 is a left lowered perspective view of the earpieces being attached together by the earpiece connector.

FIG. 7 is a perspective view of one earpiece showing an optional trim.

FIG. 8 is a side view of one earpiece.

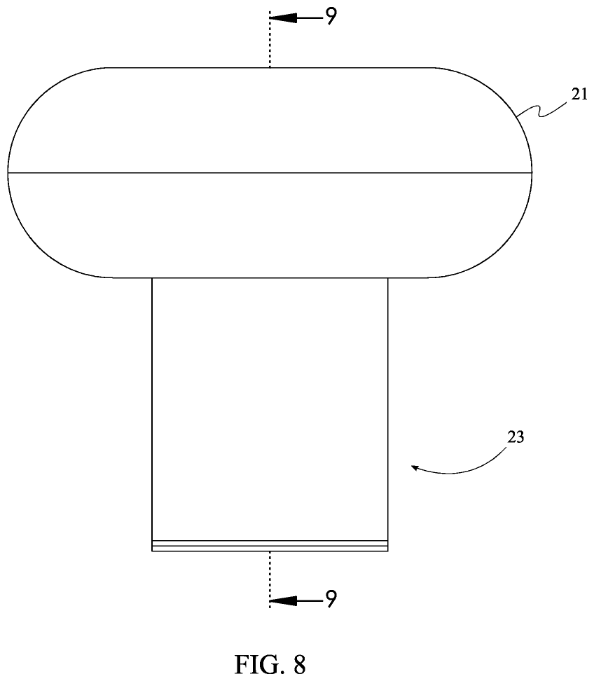

FIG. 9 is a cross-sectional view taken along line 9-9 in FIG. 8 showing a cover being connected around the base and a quantity of padding inside the base.

### DETAIL DESCRIPTIONS OF THE INVENTION

All illustrations of the drawings are for the purpose of describing selected versions of the present invention and are not intended to limit the scope of the present invention. The present invention is to be described in detail and is provided in a manner that establishes a thorough understanding of the present invention. There may be aspects of the present invention that may be practiced or utilized without the implementation of some features as they are described. It should be understood that some details have not been described in detail in order to not unnecessarily obscure focus of the invention. References herein to “the preferred embodiment”, “one embodiment”, “some embodiments”, or “alternative embodiments” should be considered to be illustrating aspects of the present invention that may potentially vary in some instances, and should not be considered to be limiting to the scope of the present invention as a whole.

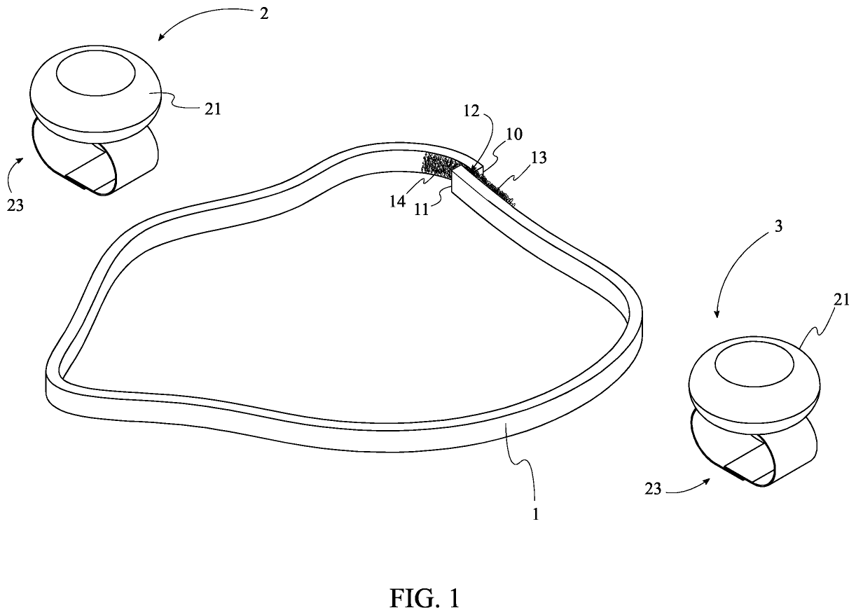

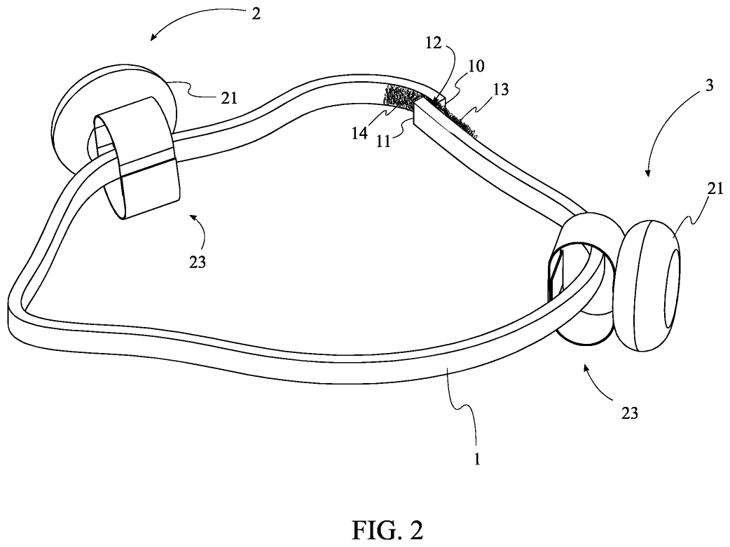

The present invention provides a protective headgear and may be known in some instances as “Hot Ears.” The intent of the present invention is to provide protection to the ears of a user from heat while undergoing a hair drying process using a hooded hair dryer. In general, referring to FIGS. 1-2, the preferred embodiment of the present invention comprises a headband 1, a first earpiece 2, and a second earpiece 3. The first earpiece 2 and the second earpiece 3 provide a structure which covers the ears of a user and protects the ears from environmental sources of discomfort such as heat from a hooded hair dryer. The headband 1 functions to position and support the first earpiece 2 and the second earpiece 3 on the user's ears. During use, the headband 1 is placed around the user's head, for example around the top of the user's head at the forehead, in order to support the first earpiece 2 and second earpiece 3 in the proper position. In the preferred embodiment, the headband 1 is constructed of a flexible material to facilitate user comfort and adjustment, though this is not necessarily a requirement.

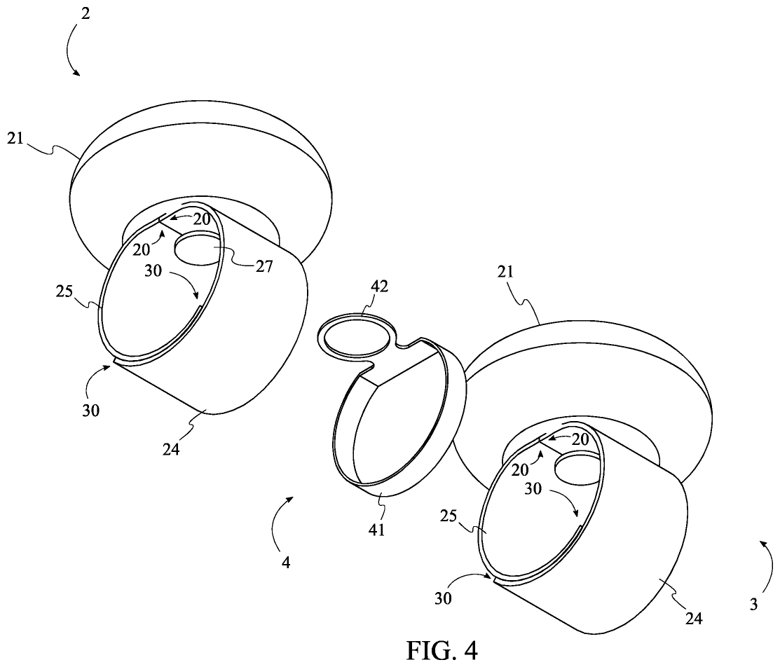

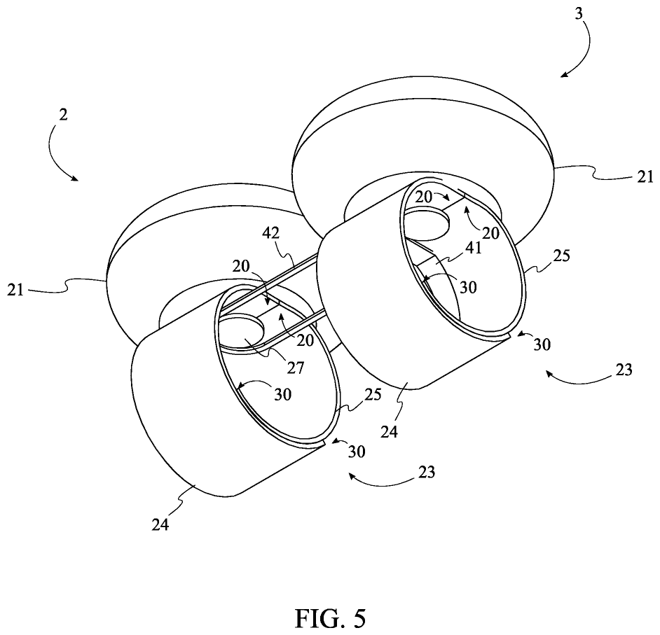

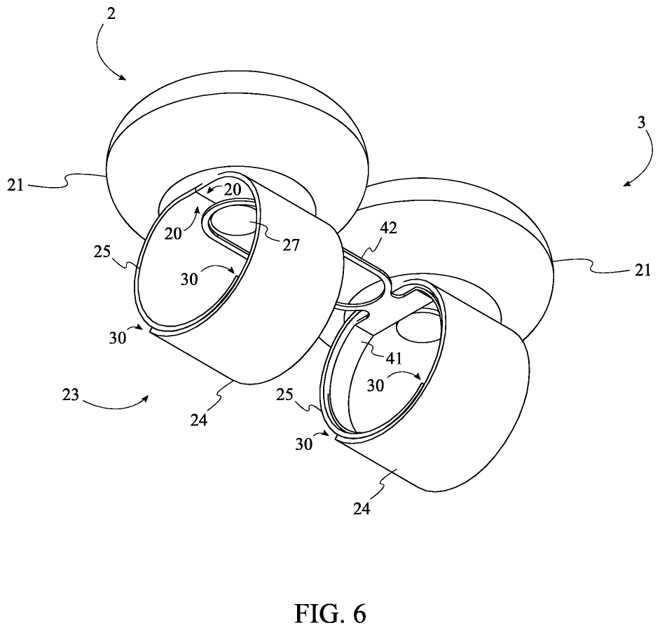

As seen in FIGS. 1-6, the first earpiece 2 and the second earpiece 3 each comprise a base 21, a cover 22, and a headband connector 23. The base 21 is the main body of the first earpiece 2 and second earpiece 3, and primarily defines the protective coverage of the present invention around the user's ears. The first earpiece 2 and the second earpiece 3 are each removably attached to the headband 1 through the headband connector 23. Furthermore, the first earpiece 2 and the second earpiece 3 are selectably positionable along the headband 1, allowing for the user to adjust the relative position of the first earpiece 2 and the second earpiece 3 to conform to their head size or for comfort reasons.

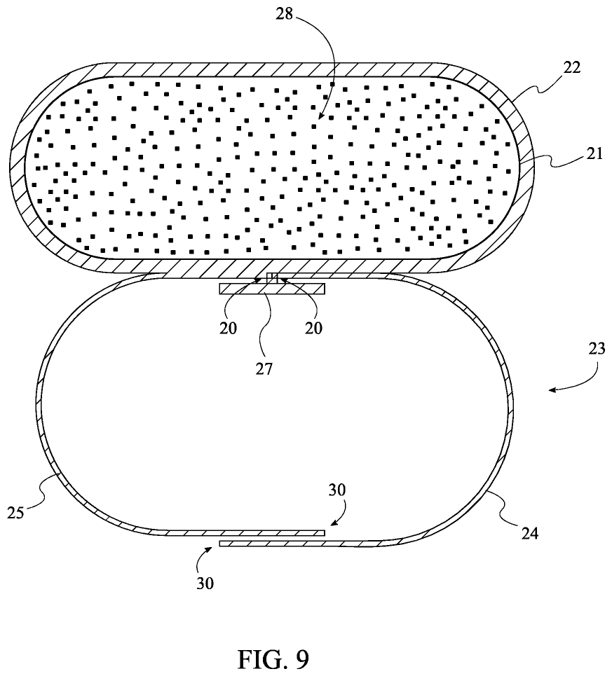

In the preferred embodiment of the present invention, the base 21 of the first earpiece 2 and the second earpiece 3 has radial geometry. More particularly, in some embodiments, the base 21 has oblate spheroidal geometry. In other embodiments, the general shape and geometry of the base 21 may vary, so long as the base 21 is capable of covering a user's ears while the present invention is worn. In various embodiments, the material construction of the base 21 may vary as desired, applicable, and/or useful. In various embodiments, the base 21 may be constructed of materials such as, but not limited to, plastic, metal, wood, foam, or any other suitable material. In some embodiments, the base 21 may be rigid. In some embodiments, the base 21 may be flexible. It is generally desirable for the base 21 to have some flexibility to enhance comfort for the user, though the base 21 should also generally maintain its shape. In the preferred embodiment, a cushion, quantity of padding 28, or similar substance is positioned within and enclosed by the base 21. The quantity of padding 28 provides internal firmness to the base 21 to enable the base 21 to maintain its desired shape, as seen in FIG. 9.

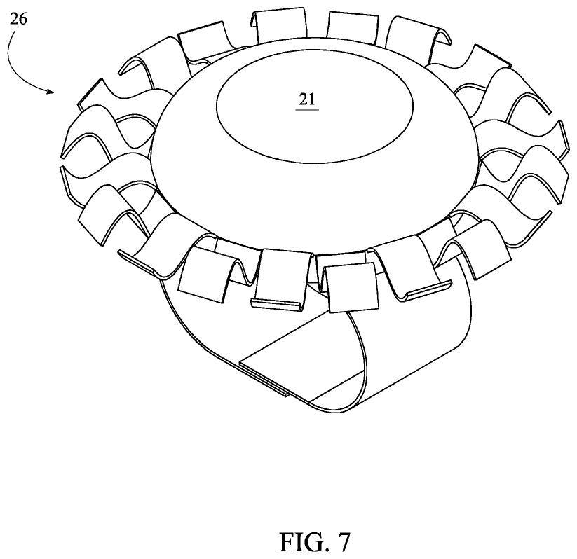

Referring again to FIG. 9, in the preferred embodiment, the cover 22 is constructed of fabric material, and is connected around the base 21 for each of the first earpiece 2 and the second earpiece 3. The cover 22 may be stitched or sewn around the base 21, or the cover 22 may be connected around the base 21 through other suitable means. The cover 22 preferably provides a soft, comfortable exterior surface to the user when the present invention is worn, and the fabric material of the cover 22 may comprise any desired color, pattern, or other visually elements to enhance the aesthetic appearance of the present invention. In the preferred embodiment, the headband 1 and the headband connector 23 of the first earpiece 2 and second earpiece 3 additionally comprise similar covers. Moreover, in some embodiments, the cover 22 may be further constructed of or comprise heat reflective material. Alternatively, a layer of heat reflective material may be connected external to the cover 22. Additionally, the first earpiece 2 and the second earpiece 3 may further comprise a trim 26 that is perimetrically connected around the base 21 for each of the first earpiece 2 and the second earpiece 3, as shown in FIG. 7. The trim 26 may be constructed of ruffled fabric or another suitable material. The trim 26 serves to embellish the aesthetic appearance of the present invention as well as partially extend the protective coverage to the user's head.

The headband connector 23 is connected adjacent to the base 21, and in the preferred embodiment is centrally positioned on the base 21 for each of the first earpiece 2 and the second earpiece 3, though the position of the headband connector 23 may vary in different embodiments as desired. The headband connector 23 facilitates removable attachment of the first earpiece 2 and the second earpiece 3 to the headband 1, in addition to allowing positional adjustment of the first earpiece 2 and the second earpiece 3 along the length of the headband 1.

In the preferred embodiment of the present invention, the headband connector 23 takes the form of a strap that is looped around the headband 1 in order to secure the earpiece to the headband 1. It should be noted, however, that other means of removably attaching the first earpiece 2 and the second earpiece 3 to the headband 1 may be utilized. For example, in some embodiments, the headband connector 23 may take the form of hook and loop tape, one or more button snaps, clips, clasps, press fit connectors, or other removable fastening means.

In the preferred embodiment, the headband connector 23 comprises a first strap portion 24 and a second strap portion 25. The first strap portion 24 and the second strap portion 25 each comprise and extend between a first end 20 and a second end 30. The first end 20 of the first strap portion 24 and the first end 20 of the second strap portion 25 are each connected to the base 21 adjacent to each other for each of the first earpiece 2 and the second earpiece 3. In order to attach the earpieces to the headband 1, the first strap portion 24 and the second strap portion 25 are looped around the headband 1 and joined together through any suitable means in order to prevent the headband 1 from being removed from the headband connector 23. In some embodiments, this may be accomplished through material properties of the first strap portion 24 and the second strap portion 25. For example, the first strap portion 24 and the second strap portion 25 may be constructed from a stiff, yet flexible material, such that the second ends 30 of the first strap portion 24 and second strap portion 25 may be positioned adjacent to each other closely enough that the headband 1 is prevented from exiting the loop formed by the first strap portion 24 and the second strap portion 25. As such, in such embodiments, a dedicated fastening means may not be necessary. However, in the preferred embodiment, the second end 30 of the first strap portion 24 and the second end 30 of the second strap portion 25 are removably attached to each other through an appropriate fastening means. For example, in various embodiments, the second end 30 of the first strap portion 24 and the second end 30 of the second strap portion 25 may be removably attached together through hook and loop tape, or through button snaps, clips, clasps, press fit connectors, or other removable fastening means. Thus, the first strap portion 24 and the second strap portion 25 are removably positioned around the headband 1, wherein the first earpiece 2 and the second earpiece 3 are removably attached to the headband 1 through the first strap portion 24 and the second strap portion 25.

In various embodiments, the particular construction of the headband 1 may vary, so long as the headband 1 is capable of securing and positioning the first earpiece 2 and the second earpiece 3 as desired on the user's head, over their ears. However, in the preferred embodiment, the headband 1 is adjustable for user comfort and to accommodate various head sizes. Preferably, as seen in FIGS. 1-2, the headband 1 comprises a first end 10, a second end 11, and a headband fastener 12, with the headband 1 extending between the first end 10 and the second end 11. The first end 10 and the second end 11 of the headband fastener 12 are removably attached to each other through the headband fastener 12. As with the previously disclosed fastening means herein, the specific nature of the headband fastener 12 may vary. However, in the preferred embodiment, the headband fastener 12 comprises a first hook and loop portion 13 and a second hook and loop portion 14. The first hook and loop portion 13 is connected adjacent to the first end 10 of the headband 1, and the second hook and loop portion 14 is connected adjacent to the second end 11 of the headband 1. The first hook and loop portion 13 may be affixed to the second hook and loop portion 14 in order to removably attach the first end 10 and the second end 11 of the headband 1 together. Moreover, the first hook and loop portion 13 and the second hook and loop portion 14 may extend a distance inward from their respective ends in order to allow for a wide range of size accommodation.

The preferred embodiment of the present invention further comprises at least one earpiece connector insert 4. The earpiece connector insert 4 may be secured to one of the other earpieces and attached to the remaining earpiece in order to attach the two earpieces together. To this end, the first earpiece 2 further comprises a fastening anchor 27. The fastening anchor 27 is connected to the base 21 of the first earpiece 2 adjacent to the headband connector 23 of the first earpiece 2. The earpiece connector insert 4 is removably positioned within the headband connector 23 of the second earpiece 3, and the earpiece connector insert 4 is removably attached to the fastening anchor 27 of the first earpiece 2.

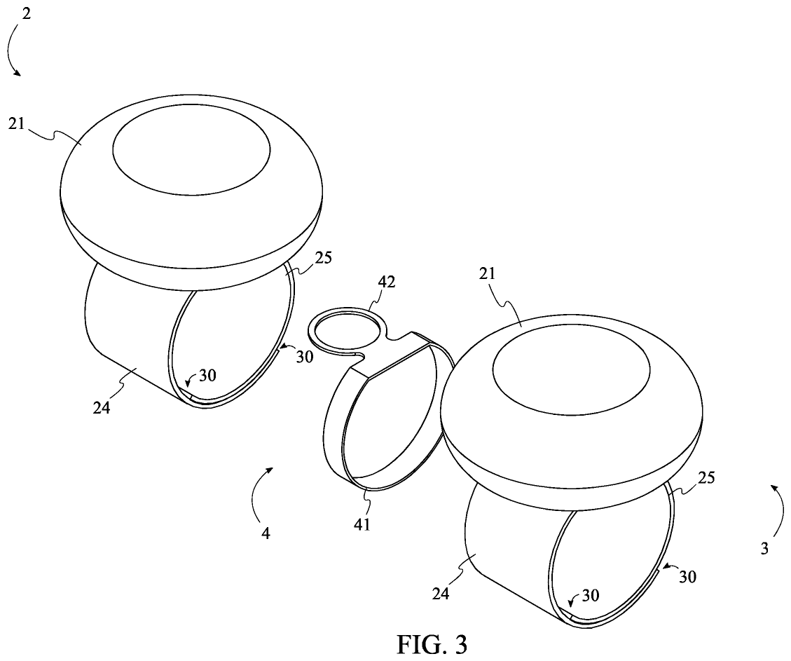

More particularly, referring to FIGS. 3-6, in the preferred embodiment, the fastening anchor 27 is a clothing button, though the specific nature of the fastening anchor 27 may vary as desired and applicable. Furthermore, the earpiece connector insert 4 comprises a connector base 41 and a loop portion 42. The loop portion 42 is perimetrically connected to the connector base 41 and is oriented perpendicular to the connector base 41. Preferably, the connector base 41 is generally circular, though this is not a strict requirement. The connector base 41 may be removably positioned within the headband connector 23 of the second earpiece 3, while the loop portion 42 is removably positioned around the fastening anchor 27 of the first earpiece 2, thus removably joining the first earpiece 2 and the second earpiece 3 together. In this configuration, the present invention may be worn over the eyes to function as a light blocker for sleeping purposes, or around the neck as an aesthetic accessory similar to a bowtie.

In the preferred embodiment of the present invention, the protective headgear comprises a plurality of earpieces, at least one connector, and a plurality of inserts. The plurality of earpieces of the protective headgear further comprises a base, a trim, and one or more straps. The base of the plurality of earpieces further comprises a front, a back, and a plurality of sides. The trim of the plurality of earpieces further comprises a first edge. The one or more straps of the plurality of earpieces further comprises a first portion, a second portion, one or more fasteners, and a button. The connector of the protective headgear further comprises a first end, a second end, an adjusting mechanism, and one or more fasteners. The plurality of inserts of the protective headgear further comprises a first section and a second section with one or more orifices.

The following is an alternative, exemplary disclosure intended to further illustrate the spirit of the present invention and is not intended to be limiting.

In the preferred embodiment of the present invention, the protective headgear comprises a plurality of earpieces. The plurality of earpieces of the protective headgear comprises a structure which covers the ears of the users and protects the ears from environment elements such as heat from a hooded hair dryer. The plurality of earpieces of the protective headgear comprises a base with a front, a back, and a plurality of sides. In the preferred embodiment of the present invention, the base of the plurality of earpieces comprises a round structure with a thickness smaller than the radius of the base.

The plurality of sides of the base receives a trim. The trim attaches to the plurality of sides of base at the first edge of the trim. In the preferred embodiment of the present invention, the trim comprises a length of material attached around the plurality of sides of the base to cover the plurality of earpieces. The trim of the plurality of earpieces can be replaced with different ornamental devices and can further be attached at different surfaces around the base of the plurality of earpieces. In alternate embodiments of the present invention, the base and trim of the plurality of earpieces can comprise a different arrangement and/or design.

In the preferred embodiment of the present invention, the plurality of earpieces of the protective headgear further comprises one or more straps. The one or more straps of the plurality of earpieces allow the plurality of earpieces to be attached to the connector of the protective headgear. The one or more straps of the plurality of earpieces is preferably provided on the back of the base of the plurality of earpieces. In the preferred embodiment of the present invention, the one or more straps of the plurality of earpieces comprises a first portion and a second portion. The first portion and second portion of the one or more straps comprises a first end which is attached to the back of the base of the plurality of earpieces. The first portion and second portion of the one or more straps further comprises a second end which can be joined together with the use of one or more fasteners. In the preferred embodiment of the present invention, the first portion and second portion of the one or more straps can be joined together utilizing one or more fasteners, such as hook and loop fasteners, provided on the second ends of the first portion and second portion. Furthermore, at least one button is provided above the first ends of the first portion and second portion of the one or more straps. The button is positioned on the back of the base above the first ends of the one or more straps to allow the plurality of earpieces to be connected together utilizing a plurality of inserts. In the preferred embodiment of the present invention, the one or more straps can further comprise a modular structure to allow the plurality of earpieces to be secured to secondary objects. In alternate embodiments of the present invention, the one or more pieces may comprise different structures to receive an additional plurality of components.

In the preferred embodiment of the present invention, the protective headgear comprises a connector. The connector of the protective headgear receives the plurality of earpieces and holds the plurality of earpieces in place on the head of the users. In the preferred embodiment of the present invention, the connector of the protective headgear comprises an elongated, thin body with a size long enough to fit around the head of the users. The body of the connector comprises a first end and a second end which can be joined together with the use of one or more fasteners. The one or more fasteners can be provided on the first end and second end of the body of the connector to allow users to join and/or detach the first end and second end of the body to/from each other. In the preferred embodiment of the present invention, the one or more fasteners of the connector can comprise a mechanism which can be easily connected and/or separated, such as a hook and loop fastener. Furthermore, the body of the connector further comprises an adjusting mechanism. The adjusting mechanism of the body of the connector allows users to adjust the length of the body of the connector to fit around the head of the users. In the preferred embodiment of the present invention, the adjusting mechanism of the connector can comprise an elastic structure integrated on the body of the connector. When a plurality of earpieces is attached to the body of the connector, the plurality of earpieces is held together. The plurality of earpieces will be held against the ears of the user thanks to the connector being wrapped around the head of the users. Thus, the ears of the users will be protected from the elements such as the heat emanated from a hooded hair dryer. In further embodiments of the present invention, the body of the connector may further comprise a mechanism to hold the plurality of earpieces in place and to prevent the plurality of earpieces from sliding along the body of the connector, such as a plurality of fasteners. In alternate embodiments of the present invention, the adjusting mechanism of the connector can comprise alternate mechanisms which allow the body of the connector to be adjusted in length.

In the preferred embodiment of the present invention, the protective headgear further comprises a plurality of inserts. The plurality of inserts of the protective headgear can be secured to the plurality of earpieces to connect multiple units of the plurality of earpieces together. In the preferred embodiment of the present invention, the plurality of inserts of the protective headgear comprises a first section and a second section. The first section of the plurality of inserts comprises a ring-like structure with a size and shape matching the one or more straps of the plurality of earpieces. The first section of the plurality of inserts is secured to the base of the plurality of earpieces utilizing the one or more straps. The first portion and second portion of the one or more straps are wrapped around the first section of the plurality of inserts and secured utilizing the one or more fasteners of the one or more straps. The second section of the plurality of inserts is provided on a side of the first section and is oriented orthogonal to the first section. In the preferred embodiment of the present invention, the second section of the plurality of inserts comprises a thin protrusion with one or more orifices. The one or more orifices of the second section of the plurality of inserts engages with the button of the one or more straps. Thus, the plurality of inserts can join a plurality of earpieces together by securing the first section of the plurality of inserts to a first earpiece and connecting the second section of the plurality of inserts to a second earpiece. In alternate embodiments of the present invention, the plurality of inserts may comprise a different structure and/or design to allow multiple units of the plurality of earpieces together.

Although the invention has been explained in relation to its preferred embodiment, it is to be understood that many other possible modifications and variations can be made without departing from the spirit and scope of the invention as hereinafter claimed.

What is claimed is:

1. An ear-protective headgear comprises:
a headband;
a first earpiece;
a second earpiece;
the first earpiece and the second earpiece each comprising a base, a cover, and a headband connector;
the cover being connected around the base for each of the first earpiece and the second earpiece;
the headband connector being connected adjacent to the base for each of the first earpiece and the second earpiece;
the first earpiece and the second earpiece each being removably attached to the headband through the headband connector; and
the first earpiece and the second earpiece being selectably positionable along the headband.

2. The ear-protective headgear as claimed in claim 1 comprises:
the base of the first earpiece and the second earpiece having radial geometry.

3. The ear-protective headgear as claimed in claim 1 comprises:
the base of the first earpiece and the second earpiece having oblate spheroidal geometry.

4. The ear-protective headgear as claimed in claim 1 comprises:
the cover being constructed of fabric material.

5. The ear-protective headgear as claimed in claim 1 comprises:
the cover being constructed of heat reflective material.

6. The ear-protective headgear as claimed in claim 1 comprises:
the headband connector being centrally positioned on the base.

7. The ear-protective headgear as claimed in claim 1 comprises:
the headband being constructed of a flexible material.

8. The ear-protective headgear as claimed in claim 1 comprises:
the headband connector being a strap.

9. The ear-protective headgear as claimed in claim 1 comprises:
the headband connector comprising a first strap portion and a second strap portion;
the first strap portion and the second strap portion each comprising a first end and a second end; and
the first end of the first strap portion and the first end of the second strap portion each being connected to the base adjacent to each other for each of the first earpiece and the second earpiece.

10. The ear-protective headgear as claimed in claim 9 comprises:
the second end of the first strap portion and the second end of the second strap portion being removably attached to each other.

11. The ear-protective headgear as claimed in claim 9 comprises:
the first strap portion and the second strap portion being removably positioned around the headband, wherein the first earpiece and the second earpiece are removably attached to the headband through the first strap portion and the second strap portion.

12. The ear-protective headgear as claimed in claim 1 comprises:
the first earpiece and the second earpiece each further comprising a trim; and
the trim being perimetrically connected around the base for each of the first earpiece and the second earpiece.

13. The ear-protective headgear as claimed in claim 1 comprises:
the headband comprising a first end, a second end, and a headband fastener;
the headband extending between the first end and the second end; and
the first end and the second end of the headband being removably attached to each other through the headband fastener.

14. The ear-protective headgear as claimed in claim 13 comprises:
the headband fastener comprising a first hook and loop portion and a second hook and loop portion;
the first hook and loop portion being connected adjacent to the first end of the headband;
the second hook and loop portion being connected adjacent to the second end of the headband; and
the first hook and loop portion being removably attached to the second hook and loop portion.

15. The ear-protective headgear as claimed in claim 1 comprises:
an earpiece connector insert;
the first earpiece further comprising a fastening anchor;
the fastening anchor being connected to the base of the first earpiece adjacent to the headband connector of the first earpiece;
the earpiece connector insert being removably positioned within the headband connector of the second earpiece; and
the earpiece connector insert being removably attached to the fastening anchor of the first earpiece.

16. The ear-protective headgear as claimed in claim 15 comprises:
the fastening anchor being a clothing button.

17. The ear-protective headgear as claimed in claim 15 comprises:
the earpiece connector insert comprises a connector base and a loop portion;
the loop portion being perimetrically connected to the connector base;
the loop portion being oriented perpendicular to the connector base;
the connector base being removably positioned within the headband connector of the second earpiece; and
the loop portion being removably positioned around the fastening anchor of the first earpiece.
